(19) United States
(12) Patent Application Publication
(10) Pub. No.: US 2009/0098173 A1
(43) Pub. Date: Apr. 16, 2009

(54) TOPICAL PAIN RELIEF PRODUCT

(76) Inventors: Ronni L. Robinson, Ambler, PA (US); David W. Wynn, Huntingdon Valley, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/249,147

(22) Filed: Oct. 10, 2008

Related U.S. Application Data
(60) Provisional application No. 60/979,222, filed on Oct. 11, 2007.

(51) Int. Cl. A61K 9/00; A61K 31/22; A61K 31/164

(52) U.S. Cl. 424/402; 424/400; 514/529; 514/613

(57) ABSTRACT

The present invention is directed to a topical composition comprising a counterirritant active ingredient and a sensate that provides a cooling sensation to the skin that is topically perceptible to an adult human subject for greater than about 90 minutes, e.g. greater than about 120 minutes, say greater than about 150 minutes, when applied in an effective amount over an area on the back of a hand, elbow, lower back or shoulder region. In one embodiment, the sensate can be encapsulated. The sensate can alternatively be bound to an ion exchange resin or adsorbed onto an adsorbant.

### CROSS REFERENCE TO RELATED APPLICATIONS

This application claims priority of the benefits of the filing of U.S. Provisional Application Ser. No. 60/979,222, filed Oct. 11, 2007. The complete disclosures of the aforementioned related U.S. patent application is/are hereby incorporated herein by reference for all purposes.

### BACKGROUND OF THE INVENTION

Topical compositions have been traditionally used for pain relief of muscles, joints, as well as skin irritation and inflammation. In recent years, other types of pain such as headache have been treated using traditional topical active ingredients such as menthol. These types of products have been available in dial-on stick and cream types of dosage forms for application to the head, even though menthol is traditionally associated with topical treatment of muscle and joint pain. These types of treatments speak to the fact that pain experiences are extremely varied in the human population, and the sources, mechanisms and treatments are not completely understood by the medical profession and consumers of pain medications.

Many traditional topical pain relief products rely on counter-irritants as active ingredients. Counter-irritants function by providing a cold, hot, tingling, or other sensation that is believed to interfere with transmission of a pain signal to the brain, providing temporary lessening of the perception of aches and pains in muscles or joints. For example, Ben Gay Ultra Strength pain relieving cream contains such counter-irritant active ingredients as menthol (10%), camphor (4%), and methyl salicylate (30%), and has an onset of sensation within about 3 minutes with about a 90 minute duration of sensation. Further, counter-irritant active ingredients provide a sensation by stimulating the TRPM8 cold receptor, or the TRPA1 cool receptor, the TRPV1 hot receptor or other receptors on the skin surface.

Additional cooling agents have been disclosed in the prior art for use as sensates, such as disclosed in U.S. Pat. No. 7,189,760, wherein a substantially pure compound and method of preparing an ethyl ester of N-[[5-methyl-2-(1-methylethyl)cyclohexyl]carbonyl]glycine is shown. This material is described as having substantially high physiological cooling activity.

Similarly, U.S. Pat. No. 7,030,273 discloses additional cooling agents. In this patent, N-alkoxyalkyl substituted-2,3-dimethyl-2-isopropylbutyramides compounds are demonstrated to have physiologic cooling effects.

### SUMMARY OF THE INVENTION

In the invention described herein it is shown that cosmetic ingredients that provide similar cooling sensations may be used to augment the pain relieving effect of the known medicinal counter-irritants. As part of the invention, traditional counter-irritants and cooling, heating or tingling sensates can be co-formulated in a multitude of pharmaceutical dosage forms. These types of ingredients also have the potential to enable use of a lower dosage of topical counter-irritants and subsequently minimize systemic absorption of irritants such as methyl salicylate.

“Cosmetic ingredient”, as used herein, refers to ingredients recognized as safe and useful for inclusion in cosmetic products, and preferably endorsed by The Cosmetic Ingredient Review. The Cosmetic Ingredient Review (CIR), based in Washington, D.C., was established in 1976 by the Cosmetic, Toiletry, and Fragrance Association (CTFA) with support of the Food and Drug Administration and the Consumer Federation of America.

“Water soluble,” as used herein in connection with non-polymeric materials, shall mean from sparingly soluble to very soluble, i.e., not more than 100 parts water required to dissolve 1 part of the non-polymeric, water soluble solute. See Remington, “The Science and Practice of Pharmacy,” pages 208-209 (2000), which is incorporated herein by reference. “Water soluble,” as used herein in connection with polymeric materials, shall mean that the polymer swells in water and can be dispersed at the molecular level to form a homogeneous dispersion or colloidal “solution”

“Active ingredient” as used herein described both the counter-irritant topical ingredient; the cooling, heating, numbing or tingling sensate; and mixtures thereof.

Counter-irritant ingredients suitable for use in the present invention include those specified in the Federal Register OTC Drug Monographs 21CFR part 348, “External Analgesic Drug Products”, including for example, Allyl isothiocryanate 0.5-5%; Methyl salicylate 10-60%; Turpentine oil 6-50% Camphor>3% to 11%; Menthol 1.25-16%; Histamine; dihydrochloride 0.025-0.10%; Methyl nicotinate 0.25-1%; Capsaicin 0.025-0.25%; Capsicum containing 0.025-0.25% capsaicin; and Capsicum oleoresin containing 0.025-0.25% capsaicin.

The present invention is directed to a topical composition comprising a cosmetic ingredient sensate that provides a cooling, warming, or tingling sensation to the skin, and optionally a counterirritant active ingredient. The topical composition provides a cooling, warming, or tingling sensation to the skin that is topically perceptible to an adult human subject for greater than about 90 minutes, e.g. greater than about 120 minutes, say greater than about 150 minutes, when applied in an effective amount over an area on the back of a hand, elbow, lower back or shoulder region. In one embodiment, the sensate can be encapsulated. The sensate can alternatively be bound to an ion exchange resin or adsorbed onto an adsorbant.

The cosmetic ingredient sensate can be selected from the group consisting of [(−)-isopulegol, (2S)-3-(1-menthoxy)propane-1,2-diol, “Frescolat MGA”/menthone glycerin acetal, “Frescolat ML”/menthyl lactate, “WS-14”/N-t-butyl-p-menthane-3-carboxamide, “WS-23”/2-Isopropyl-N,2,3-trimethylbutyramide, WS-12/N-(4-methoxyphenyl)-p-menthane-3-carboxamide, “WS-3”/N-Ethyl-p-menthane-3-carboxamide, and “WS-5”/Ethyl 3-(p-menthane-3-carboxamido)acetate].

In one embodiment, the topical composition has resistance to wash-off and/or a skin adhesion sufficient to withstand tape stripping.

In one embodiment, the topical composition has a re-activation feature wherein the sensate is encapsulated such that rubbing the product refreshes the sensory effect by making more sensate ingredient available to the skin.

In another embodiment, the topical composition has re-activation feature wherein moisture, for example from perspiration, refreshes the sensory effect by making more sensate ingredient available to the skin.

The present invention is also directed to an occlusive patch for prolonged contact with the skin incorporated with the topical composition described above.

The present invention also relates to a sprayed on film or fabric that incorporates the topical compositions described above.

### DETAILED DESCRIPTION OF THE INVENTION

The compositions of the present invention may be prepared in a number of forms for application to the skin (topical application) of a patient. For example, the composition may be applied in a gel, cream, ointment, liquid, spray liquid, paint-/brush-on preparation, solidifying emulsion or cream (i.e. facial mask), aerosol, powder, oil, balm, salve, or adhesive bandage. The topical compositions of the present invention may be in the form of meltable solids, semi-solids, solutions, suspensions, or emulsions. In addition the composition may be impregnated on a bandage, hydrocolloid dressing, treatment patch or on cloth wipe products. In one embodiment, the topical composition may have resistance to moisture and subsequent washing off of the skin.

In certain embodiments, the topical compositions of the present invention comprise a dermatologically acceptable carrier. Such a carrier is suitable for topical use that is compatible with the active ingredients described herein. An effective and safe carrier varies from about 50% to about 99% by weight of the compositions of this invention, more preferably from about 75% to about 99% of the compositions and most preferably from about 75% to about 95% by weight of the compositions.

The topical compositions useful in the present invention can be formulated as solutions. Solutions typically include an aqueous or organic solvent (e.g., from about 50% to about 99.99% or from about 90% to about 99% of a cosmetically acceptable aqueous or organic solvent). Examples of suitable organic solvents include:

propylene glycol, polyethylene glycol (200-600), polypropylene glycol (425-2025), glycerol, 1,2,4-butanetriol, sorbitol esters, 1,2,6-hexanetriol, ethanol, and mixtures thereof.

Topical compositions useful in the subject invention may be formulated as a solution comprising an emollient. Such compositions preferably contain from about 2% to about 50% of an emollient(s). As used herein, “emollients” refer to materials used for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972) and the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7th Edition, 1997) (hereinafter “ICI Handbook”) contains numerous examples of suitable materials.

A lotion can be made from a solution containing an emollient. Lotions typically comprise from about 1% to about 20% (e.g., from about 5% to about 10%) of an emollient(s) and from about 50% to about 90% (e.g., from about 60% to about 80%) of water. Another type of product that may be formulated from a solution containing an emollient is a cream. A cream typically comprises from about 5% to about 50% (e.g., from about 10% to about 20%) of an emollient(s) and from about 45% to about 85% (e.g., from about 50% to about 75%) of water.

Yet another type of product that may be formulated from a solution containing an emollient is an ointment. An ointment can comprise a simple base of animal or vegetable oils or semi-solid hydrocarbons. An ointment may comprise from about 2% to about 10% of an emollient(s) plus from about 0.1% to about 2% of a thickening agent(s). A more complete disclosure of thickening agents or viscosity increasing agents useful herein can be found in Sagarin, Cosmetics, Science and Technology, 2nd Edition, Vol. 1, pp. 72-73 (1972) and the ICI Handbook pp. 1693-1697.

The topical compositions useful in the present invention can be formulated as emulsions. If the carrier is an emulsion, from about 1% to about 10% (e.g., from about 2% to about 5%) of the carrier comprises an emulsifier(s). Emulsifiers may be nonionic, anionic or cationic. Suitable emulsifiers are disclosed in, for example, U.S. Pat. Nos. 3,755,560, 4,421,769, McCutcheon's Detergents and Emulsifiers, North American Edition, pp. 317-324 (1986), and the ICI Handbook, pp. 1673-1686.

Lotions and creams can be formulated as emulsions. Typically such lotions comprise from 0.5% to about 5% of an emulsifier(s). Such creams would typically comprise from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the cosmetic art and are useful in the subject invention. Multi-phase emulsion compositions, such as the water-in-oil-in-water type, as disclosed in U.S. Pat. Nos. 4,254,105 and 4,960,764, are also useful in the subject invention. In general, such single or multiphase emulsions contain water, emollients, and emulsifiers as essential ingredients.

The topical compositions of this invention can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprises between about 0.1% and 5%, by weight, of such gelling agents.

The topical compositions of the present invention can also be formulated into a solid delivery system (e.g., a wax-based stick, soap bar composition, powder, or a wipe containing powder).

Liposomal formulations are also useful compositions of the subject invention. In one embodiment, the peptide and/or the pigment are contained within the liposome. Examples of liposomes are unilamellar, multilamellar, and paucilamellar liposomes, which may or may not contain phospholipids. Such compositions can be prepared by first combining hesperetin with a phospholipid, such as dipalmitoylphosphatidyl choline, cholesterol and water according to the method described in Mezei & Gulasekharam, “Liposomes—A Selective Drug Delivery System for the Topical Route of Administration; Gel Dosage Form”, Journal of Pharmaceutics and Pharmacology, Vol. 34 (1982), pp. 473-474, or a modification thereof. Epidermal lipids of suitable composition for forming liposomes may be substituted for the phospholipid. The liposome preparation may then be incorporated into one of the above carriers (e.g., a gel or an oil-in-water emulsion) in order to produce the liposomal formulation. Other compositions and uses of topically applied liposomes are described in Mezei, M., “Liposomes as a Skin Drug Delivery System”, Topics in Pharmaceutical Sciences (D. Breimer and P. Speiser, eds.), Elsevier Science Publishers B. V., New York, N.Y., 1985, pp. 345-358, PCT Patent Application No. WO96/31194, Niemiec, et al., 12 Pharm. Res. 1184-88 (1995), and U.S. Pat. No. 5,260,065.

In one-embodiment, the liposome is non-ionic. In one example, the liposome contains (a) glycerol dilaurate; (b) compounds having the steroid backbone found in cholesterol; and (c) fatty acid ethers having from about 12 to about 18 carbon atoms. In a further embodiment, the liposome comprises glycerol dilaurate, cholesterol, polyoxyethylene-10-stearyl ether, and polyoxyethylene-9-lauryl ether. In one embodiment, these ingredients are in a ratio of about 38:12:33:17.

In one embodiment, the liposomes are present in the topical composition in an amount, based upon the total volume of the composition, of from about 10 mg/ml to about 100 mg/ml such as from about 15 mg/ml to about 50 mg/ml. Methods of preparing liposomes are well known in the art.

The topical compositions useful in the subject invention may contain, in addition to the aforementioned components, a wide variety of additional oil-soluble materials and/or water-soluble materials conventionally used in compositions for use on skin, hair, and nails at their art-established levels.

Various other materials may also be present in the compositions useful in the subject invention. These include adsorbants, humectants, proteins and polypeptides, preservatives and an alkaline agent. Examples of such agents are disclosed in the ICI Handbook, pp. 1650-1667.

The compositions of the present invention may also comprise chelating agents (e.g., EDTA) and preservatives (e.g., parabens). Examples of suitable preservatives and chelating agents are listed in pp. 1626 and 1654-55 of the ICI Handbook. In addition, the topical compositions useful herein can contain conventional cosmetic adjuvants, such as dyes, opacifiers (e.g., titanium dioxide), pigments, and fragrances.

In one embodiment the composition is delivered in a stick or bar which is rubbed upon the skin wherein the active ingredient is deposited on the skin. In one embodiment the sensate and counter-irritant or other active ingredients are impregnated into in a patch or fabric for prolonged contact with the skin. In one embodiment the active ingredient is incorporated into a pumpable or sprayable solution, suspension, or emulsion which forms a continuous or discontinuous coating, an adhesive film, or a nonwoven fabric when sprayed onto the skin. In one such embodiment, the active ingredient(s) are incorporated into a polymer-containing solution, suspension, or emulsion, which forms a solid mesh or entrapment upon application which adheres to the skin and solidifies.

In one particular embodiment, the active ingredient(s) are incorporated into a sprayable composition comprising fibers, a binder, and a diluent. In this embodiment, the sprayable composition may be in the form of a suspension of small fibers homogeneously mixed in a suitable diluent, together with a binder. The fibers may be synthetic or natural materials, such as cotton, wool, silk, cashmere, linen seaweed cellulose, ramie cellulose, mink fur, rabbit hair, aramid, chitosan, carbon, glass, metal, ceramic, or other fibers, in dimensions sufficiently small to flow through a nozzle. The diluent may be any suitable solvent, such as acetone, water, ethyl acetate, or the like. The binder may be any suitable polymer or mixture of polymers that is soluble in the diluent, and solid at room temperature, such as polyvinyl acetate, polyvinyl butyrate, polyvinyl alcohol, and natural latex. The binder aids with adhesion of the sprayed fibers to one another. Preferably the fibers have a length about 20 microns to about 200 microns, and a length to diameter ratio of at least about 3:1. One such suitable sprayable composition is disclosed in published PCT application WO 03/104540, which is incorporated herein by reference.

In another embodiment, the active ingredient(s) are incorporated into a cream or lotion vehicle such as for example the type disclosed in U.S. Patent No. 6,284,234. A preferred embodiment comprises a) from about 1 percent to about 10 percent of a nonionic lipid; b) from about 75 percent to about 98 percent of a vehicle solution comprised of water or a mixture of water and a hydrophilic compound and a second vehicle component comprised of an alcohol, a polyol, or mixtures thereof, and c) an effective amount of the active ingredient(s).

In another embodiment, the active ingredient(s) are incorporated into a cream or lotion vehicle having superior adhesion to the skin or superior resistance to wash-off by water or perspiration. In certain such embodiments, the topical composition may comprise one or more hydrophobic polymers. Active ingredient skin adhesion can be measured by means of tape stripping. U.S. Pat. No. 6,924,256, for example, describes a tape stripping procedure wherein suitable tape, e.g. white sellotape, is placed on skin under controlled pressure for 2 minutes and gently removed. Typically two sequential tape strips are collected from each sampling site. The tapes are analyzed for active ingredients.

U.S. Pat. No. 7,097,828 also describes a procedure wherein sunscreen products are applied to skin. At defined time points, the sunscreen treated sites were tape-stripped six consecutive times using 3M Highland invisible Tape. The tape was 1.9 cm wide or about 5 times the width of the sunscreen application. Each tape strip was placed separately in a 25 mL scintillation vial and left to soak overnight in isopropyl alcohol. After soaking overnight, an aliquot of the isopropyl alcohol was removed and the sunscreen level measured using a UV-Vis Spectrophotometer. The amount of sunscreen recovered from each tape strip was calculated and then levels determined from each of the six tape strips were summed to calculate the total amount of sunscreen recovered from the skin at each defined time point. Mean recoveries of sunscreen from skin tape-stripped either immediately or after 4 hours are reported as a mean from 4 different individuals.

A suitable wash-off resistant vehicle is disclosed, for example, in published PCT Application WO 2005/070371. In this example, the topical composition comprises a watertight complex at a level of about around 0.5-1%. The watertight complex comprises a blend of acrylates/octylacrylamide copolymer, and hydrolyzed Jojoba esters in a ratio of about 2:1, (and) water. A preferred copolymer in that application is an acrylate octylacrylamide copolymer with an acidity of 2.4 meq/g. Such a product is commercially available as DERMACRYL AQF or DERMACRYL 79 from National Starch Corporation. Another preferred in that case is hydrolyzed jojoba ester that is available as FLORAESTERS K-20W Jojoba by Int. Floratech Technology. Ltd., Hartsdale, N.Y., USA.

Another suitable wash-off resistant vehicle is disclosed, for example in U.S. Pat. No. 6,756,059. In such embodiments, the topical pain relief composition may comprise a topical composition precursor, together with a suitable amount of diluent, such as for example water. When provided as a use solution, the topical composition has an ability to adhere or bind to skin tissue and thereby hold active ingredients in proximity to skin tissue. In addition, the topical composition has an ability to hold or contain active ingredients so that the active ingredients can be made available to skin tissue when the topical composition is applied to skin tissue. Active ingredients that can be used include natural and synthetic substances that produce a desired effect when placed on skin tissue and may include medicines or drugs or other substances intended for the diagnosis, cure, mitigation, treatment, or prevention of a disease or condition, and in particular for treatment of pain, and may include substances that may be characterized as protectants, repellants, and moisturizers.

The topical composition precursor of this embodiment can be provided as a result of melt processing a hydrophobic polymer composition and a hydrophilic polymer composition in the presence of less than about 1% by weight water. The hydrophobic polymer composition includes a poly (vinylpyrrolidone/alkylene) polymer wherein the alkylene group contains at least about 10 carbon atoms. The hydrophilic polymer composition includes at least one of a hydrophilic polymer comprising repeating carboxylic acid groups and/or repeating hydroxyl groups. Exemplary hydrophilic polymers include polyacrylic acid having a weight average molecular weight of at least about 50,000 and exhibiting less than 1% cross-linking, poly (maleic acid/methylvinylether) copolymer having a weight average molecular weight of at least about 50,000, starch, derivatives of starch, cellulose, derivatives of cellulose, carboxymethyl cellulose, polyvinyl alcohol, cyclodextrins, dextrans, and mixtures thereof. The hydrophilic polymer composition can include polyacrylic acid having a weight average molecular weight of between about 50,000 and about 4,000,000, and exhibits less than 1% cross-linking and/or poly(maleic acid/methylvinylether)copolymer having a weight average molecular weight of between about 50,000 and about 4,000,000.

The hydrophobic polymer composition can include a mixture of different poly(vinylpyrrolidone/alkylene)polymers. When the hydrophobic polymer composition contains a mixture of two different poly(vinylpyrrolidone/alkylene)polymers, the first poly(vinylpyrrolidone/alkylene)polymer can be provided at a concentration of between about 5 wt. % and about 54 wt. %, based on the weight of the hydrophobic polymer composition. In addition, the second poly(vinylpyrrolidone/alkylene)polymer can be provided at a concentration of between about 46 wt. % and about 95 wt. %, based on the weight of the hydrophobic polymer composition. Exemplary poly(vinylpyrrolidone/alkylene)polymers include poly(vinylpyrrolidone/1-eicosene)polymer and poly(vinylpyrrolidone/hexadecene).

The topical composition precursor can be formed by mixing the hydrophobic polymer composition and the hydrophilic polymer composition in a melt and providing a functional group parity between the pyrrolidone groups of the hydrophobic polymer composition and the combination of carboxylic acid groups and/or hydroxyl groups of the hydrophilic polymer composition that is between about 1:1 and about 5:1, and can be between about 1.5:1 and about 3:1. For certain compositions, it is expected that this functional group parity of the hydrophobic polymer composition to the hydrophilic polymer composition will result in a topical composition precursor containing about 72 wt. % to about 98 wt. % hydrophobic polymer composition and about 2 wt. % to about 25 wt. % hydrophilic polymer composition, based on the total weight of the topical composition precursor.

The topical composition can include the topical composition precursor and can include a result of diluting the topical composition precursor with water. The topical composition preferably includes a result of hydrating the topical composition precursor with water to provide at least about 30 wt. % water. The topical composition can be characterized as a concentrate if it contains between about 30 wt. % and about 70 wt. % water based on the weight of the topical composition. It is expected that the concentrate will be provided with a water concentration of between about 30 wt. % and about 45 wt. % to reduce costs associated with shipping water. When the topical composition is provided as a use solution for application to skin tissue, it is expected that the composition will contain at least about 70 wt. % water and can include between about 70 wt. % and about 96 wt. % water, based on the weight of the topical composition.

The topical composition may be manufactured by a method that includes a step of melt processing a mixture of a hydrophobic polymer composition and a hydrophilic polymer composition to provide a topical composition precursor, and diluting the topical composition precursor to provide a concentrate having a water concentration of at least about 30 wt. %, based on the weight of the topical composition. The step of melt processing preferably includes mixing the hydrophobic polymer composition and the hydrophilic polymer composition at a temperature of greater than 50° C., and more preferably greater than about 125° C. The step of melt processing preferably includes mixing the hydrophobic polymer composition and the hydrophilic polymer composition at a water concentration of less than about 1 wt. %.

Compositions that exhibit wash-off resistance are known. For example, published U.S. Patent Application No. 2005/0232876 describes skin care compositions that are useful as cosmetic, protective and therapeutic dermatological compositions that exhibit smoothness and water resistance when applied to the skin. In examples, the contact angle of water on films of the inventive compositions was measured to show the water resistant nature of these mixtures. Contact angle is a measure of the surface wettability and is described in Test Method ASTM D5725-99.

Published U.S. Patent Application 2005/0267210, which is incorporated herein by reference, asserts that dimethicone, a variety of polydimethylsiloxanes (less volatile) and the cyclomethicones have a long history of use in cosmetic preparations, and as vehicles, they allow good spreading of actives on the skin and will eventually evaporate. They are insoluble in water, so that resistance to water wash-off of actives is imparted. The cyclomethicones can be turned into gels for ease of application to the skin.

Published U.S. Patent Application 2006/0064068, which is incorporated herein by reference, describes film forming barrier compositions that form a thin film coating over the skin which is resistant to wash-off by water or body fluids. These compositions incorporate film-forming agents that are preferably soluble or miscible with oleogenous components in the composition to provide a substantially homogeneous mixture. Thus suitable film-forming agents are preferably oleophilic and water-resistant.

Published U.S. Patent Application 2006/0188459, which is incorporated herein by reference, describes cosmetic, pharmaceutical or dermatological preparations comprising copolymer waxes. With the aid of the waxes taught therein, the water resistance of cosmetic products, for example sunscreen compositions, can be increased.

Published U.S. Patent Application 2005/0287097, which is incorporated herein by reference, describes a test for wash-off resistance that utilizes hydrophilic nylon membranes normally used for solvent filtration. It is expected that these membranes possess mixed hydrophilic and hydrophobic properties similar to skin.

In one embodiment the topical pain relief composition comprises a combination of an FDA monograph counter-irritant active ingredient with a microencapsulated sensate. In one embodiment the counter-irritant active ingredient is microencapsulated.

In one embodiment the counter-irritant or sensate is microencapsulated with a polymer or coating system that ruptures upon rubbing, providing a re-activation of the cooling or heating sensation, providing a longer lasting formula or formula with a refresh ability. In one embodiment the coating system for a reactivating ingredient able to release upon rubbing include hydrocolloids.

Examples of suitable hydrocolloids (also referred to herein as gelling polymers) include but are not limited to gelatin, alginates, agar, guar gum, locust bean, carrageenan, tara, gum arabic, tragacanth, pectin, xanthan, gellan, maltodextrin, galactomannan, pusstulan, laminarin, scleroglucan, gum arabic, inulin, pectin, whelan, rhamsan, zooglan, methylan, chitin, chitosan, and derivatives and mixtures thereof. In certain embodiments wherein the counter-irritant or sensate is in the form of an oil, lipid or fatty acid it may be advantageous to adsorb onto a suitable substrate or adsorbant prior to coating or microencapsulation.

In another embodiment the counter-irritant or sensate is microencapsulated in a liquid or oil state, by a combination of a hydrocolloid and a film forming polymer. Hydrocolloids are particularly useful in this embodiment since they are flexible and accommodate liquid materials. In one embodiment the coating functions to prevent the active ingredient from dissolving in the lotion or cream formulation carrier.

In one embodiment the reactivation of the active monograph counterirritant or sensate occurs when in the presence of moisture, either from the environment, or from the skin, e.g. from perspiration. In such an embodiment the sensate is coated as a particle or particulate, utilizing coating systems that are sensitive to moisture immediately or over time. In such a system, the polymer may be dissolved immediately or hydrated slowly over time using polymer systems such as those comprising water soluble film forming polymers.

Examples of suitable water soluble film formers include, but are not limited to, polyvinylalcohol (PVA), polyvinylpyrrolidone (PVP), hydroxypropyl starch, hydroxyethyl starch, pullulan, methylethyl starch, carboxymethyl starch, methylcellulose, hydroxypropylcellulose (HPC), hydroxyethylmethylcellulose (HEMC), hydroxypropylmethylcellulose (HPMC), hydroxybutylmethylcellulose (HBMC), hydroxyethylethylcellulose (HEEC), hydroxyethylhydroxypropylmethyl cellulose (HEMPMC), methacrylic acid and methacrylate ester copolymers, polyethylene oxide and polyvinylpyrrolidone copolymers, gelatin, proteins such as whey protein, coagulatable proteins such as albumin, casein, and casein isolates, soy protein and soy protein isolates, pre-gelatinized starches, and polymers and derivatives and mixtures thereof.

In certain embodiments the polymer system comprises a combination of an insoluble polymer and a pore forming water-soluble material. The water-soluble material may include water-soluble polymers such as hypromellose, hydroxypropyl cellulose, polyvinylpyrrolidone (PVA), and methacrylic acid and methacrylate ester copolymers. Suitable water insoluble film-forming polymers include, but are not limited to, cellulose esters, cellulose ethers, cellulose ester-ethers, polyvinyl alcohols, polyvinyl acetate, polycaprolactones, polyacrylates, polymethacrylates, and derivatives, copolymers, and combinations thereof.

In one embodiment the reactivation of the active monograph counter-irritant or sensate occurs when in the presence of moisture, either from the environment, or from the skin, e.g. from perspiration. In such an embodiment the counter-irritant is coated as a particle or particulate, utilizing coating systems which are sensitive to moisture immediately or over time.

Particles may be coated or encapsulated with the coating systems described above through a coacervation process a fluid bed process. Such coacervation-encapsulated active ingredients are commercially available from, for example, Eurand America, Inc. or Circa Inc. coating may be applied to the active ingredient particle cores via any suitable method known in the art. Additional suitable coating methods include high sheer granulation, fluid bed granulation, e.g. rotor granulation, fluid bed coating, wurster coating, coacervation, spray drying, spray congealing, and the like and are described in, for example, Pharmaceutical Dosage Forms: Tablets Volume 3, edited by Herbert A. Lieberman and Leon Lachman, Chapters 2, 3, and 4 (1982).

In one embodiment the coating level sufficient for reactivation upon rubbing is greater than 10 percent by weight of the sensate, e.g. greater than 20 percent by weight of the sensate. In one embodiment the coating level sufficient for reactivation through the release of moisture, for example through perspiration, is greater than 10 percent by weight of the sensate, e.g. greater than 20 percent by weight of the sensate.

In one embodiment the active monograph counter-irritant or sensate ingredient is bound or complexed to an ion exchange resin, wherein release of the active ingredient is dependent on the salt available in the perspiration of a user. In this embodiment the metal ions in the perspiration are exchanged with the pre-bound active resinate, thereby releasing the active monograph counter-irritant or sensate ingredient. In this embodiment the release of the ingredient occurs over time. As used herein “drug-resin complex” shall mean the bound form of any of the active monograph counter-irritant or sensate ingredients.

The drug-resin complex is also referred to in the art as a “resinate”. An example of a suitable ion exchange resin for NSAID active ingredients includes, but is not limited to, styrene/divinyl benzene copolymers and cholestyramines, which are commercially available from Rohm & Haas under the tradename, “Duolite® AP143.” An example of a suitable ion exchange resin for positively charged active ingredients, includes, but is not limited to, a sulfonic acid cationic ion exchange resin derived from a sulfonated styrene/divinyl benzene copolymer, such as those commercially available from Rohm & Haas under the general tradename “Amberlite,” e.g., “Amberlite IRP69,” and those commercially available from Dow Chemical Company, sold under the tradename, “Dowex,” e.g., “Dowex Marathon,” “Dowex Monosphere,” and “Dowex XYS-40010.00.”

Ion exchange resins are generally classified into various types, including strong acid cations, strong base cations, weak acid cations and weak base cations. In general, the active is mixed with an aqueous suspension of a suitable resin, and the resin-active complex is then washed and dried. Binding of the drug onto the resin may be demonstrated by analyzing the pH of the media eluting from the wash or by measuring a change in sodium concentration of the wash.

In one embodiment the active monograph topical counter-irritant or the sensate may be adsorbed onto a suitable carrier. Suitable adsorbants may include trisilicates such as but not limited to magnesium aluminate metasilicate and magnesium aluminometasilicate, clays, vermiculite, agglomerated maltodextrin, tribasic calcium phosphate or dibasic calcium phosphate, silica gels, and silicified microcrystalline cellulose. In such an embodiment the adsorbed active ingredient may be further released upon rubbing onto the skin.

Topically applied pain relieving composition comprising combinations of non-monograph cosmetic ingredient sensates (plus optionally menthol or other monograph ingredient) having pain relieving and cooling effects for greater than about 90 minutes, e.g. greater than about 120 minutes, say greater than about 150 minutes. Said compositions preferably comprise a sensate material selected from [(−)-isopulegol, (2S)-3-(1 menthoxy)propane-1,2-diol, “Frescolat MGA”/menthone glycerin acetal, “Frescolat ML”/menthyl lactate, “WS-14”/N-t-butyl-p-menthane-3-carboxamide, “WS-23”/2 Isopropyl-N,2,3-trimethylbutyramide, WS-12/N-(4-methoxyphenyl)-p-menthane-3 carboxamide, “WS-3”/N-Ethyl-p-menthane-3-carboxamide, and “WS-5”/Ethyl 3-(p-menthane-3-carboxamido)acetate].

In one embodiment the encapsulated sensate or encapsulated counter-irritant coating must have sufficient tensile strength to allow for an initial application of the composition, but also retain integrity such that when the composition is re-rubbed at a later time, a portion of the active is re-released on the skin. In this embodiment greater than 20 percent, e.g. greater than 50 percent of the encapsulated sensate retains its coating integrity when measured using a Scanning Electron Microscope, after initial application of up to 2 minutes.

The present invention includes the sustained activity of all types of counter-irritants FDA monograph topical active ingredients, including sensates which may not be indicated in the monograph for pain relief, also those that produce a “hot” or “tingling” sensation, in addition to those that work to activate the cold receptor, as all types of these sensory signals can interrupt the perception of the pain signal.

One composition according to the present invention contains 10% menthol, and about 2% to about 20% of a mixture of non-monograph cosmetic ingredients selected from: (−)-isopulegol, (2S)-3-(1-menthoxy)propane-1,2-diol, “Frescolat MGA”/menthone glycerin acetal, “Frescolat ML”/menthyl lactate, “WS-14”/N-t-butyl-p-menthane-3-carboxamide, “WS-23”/2-Isopropyl-N,2,3-trimethylbutyramide, WS-12/N-(4-methoxyphenyl)-p-menthane-3-carboxamide, “WS-3”/N-Ethyl-p-menthane-3-carboxamide, and “WS-5”/Ethyl 3-(p-menthane-3-carboxamido)acetate.

Suitable external analgesics include but are not limited to those disclosed in the Tentative Final Monograph for External Analgesic Drug Products for over-the-counter human use, U.S. Federal Register Vol. 48, No. 27, Feb. 28, 1983. These monographed external analgesics include counter-irritants that produce redness, for example, Allyl isothiocryanate 0.5-5%, Methyl salicylate 10-60%, and Turpentine oil 6-50%; Irritants that produce cooling, for example, Camphor>3% to 11%, or Menthol 1.25-16%; Irritants that produce vasodilation, for example Histamine dihydrochloride 0.025-0. 10%, or Methyl nicotinate 0.25-1%; and irritants that do not produce redness, for example, Capsaicin 0.025-0.25%, Capsicum containing 0.025-0.25% capsaicin, or Capsicum oleoresin containing 0.025-0.25% capsaicin.

Suitable non-monograph cosmetic ingredient sensates are selected from the group including but are not limited to [(−)-isopulegol, (2S)-3-(1-menthoxy)propane-1,2-diol, “Frescolat MGA”/menthone glycerin acetal, “Frescolat ML”/menthyl lactate, “WS-14”/N-t-butyl-p-menthane-3-carboxamide, “WS-23”/2-Isopropyl-N,2,3-trimethylbutyramide, WS-12/N-(4-methoxyphenyl)-p-menthane-3-carboxamide, “WS-3”/N-Ethyl-p-menthane-3-carboxamide, and “WS-5”/Ethyl 3-(p-menthane-3-carboxamido)acetate].

1. A topical composition comprising a counterirritant active ingredient and a cosmetic ingredient sensate that provides a cooling, warming, or tingling sensation to the skin that is topically perceptible to an adult human subject for greater than about 90 minutes, e.g. greater than about 120 minutes, say greater than about 150 minutes, when applied in an effective amount over an area on the back of a hand, lower back or shoulder.

2. The topical composition of claim 1 wherein the sensate is encapsulated.

3. The topical composition of claim 1 wherein the sensate is bound to an ion exchange resin.

4. The topical composition of claim 1 wherein the sensate is adsorbed onto an adsorbant.

5. The topical counterirritant composition of claim 1 wherein the sensate is selected from the group consisting of [(−)-isopulegol, (2S)-3-(1-menthoxy)propane-1,2-diol, “Frescolat MGA”/menthone glycerin acetal, “Frescolat ML”/menthyl lactate, “WS-14”/N-t-butyl-p-menthane-3-carboxamide, “WS-23”/2-Isopropyl-N,2,3-trimethylbutyramide, WS-12/N-(4-methoxyphenyl)-p-menthane-3-carboxamide, “WS-3”/N-Ethyl-p-menthane-3-carboxamide, and “WS-5”/Ethyl 3-(p-menthane-3-carboxamido)acetate].

6. A topical composition of claim 1 having a skin adhesion sufficient to withstand tape stripping.

7. A topical composition of claim 1 comprising a re-activation feature wherein the sensate is encapsulated such that rubbing the product refreshes the sensory effect by making previously encapsulated sensate ingredient available to the skin.

8. A topical composition of claim 1 comprising a re-activation feature wherein moisture, for example from perspiration, refreshes the sensory effect by making more sensate ingredient available to the skin.

9. An occlusive patch for prolonged contact with the skin incorporated with the composition of claim 1.

10. A sprayed on film or fabric incorporated with the composition of claim 1.
